(19) United States
(12) Patent Application Publication
(10) Pub. No.: US 2003/0059413 A1
(43) Pub. Date: Mar. 27, 2003

(54) RETROVIRAL VECTORS ENCODING INTERFERON ALPHA AND USES THEREOF

(76) Inventors: NADER G. ABRAHAM, VALHALLA, NY (US); TAUSEEF AHMED, VALHALLA, NY (US)

Correspondence Address: FULBRIGHT & JAWORSKI, LLP, 666 FIFTH AVE, NEW YORK, NY, 10103-3198, US

(21) Appl. No.: 09/435,538

(22) Filed: Nov. 8, 1999

Related U.S. Application Data
(60) Provisional application No. 60/107,887, filed on Nov. 9, 1998.

(51) Int. Cl. A61K 48/00; A61K 38/21; C12P 21/04; C12N 5/06

(52) U.S. Cl. 424/93.21; 424/85.7; 514/44; 530/351; 435/320.1; 435/325; 435/365.1; 435/70.5

(57) ABSTRACT

Retroviral vectors which encode interferon alpha are disclosed. These canbe used to produce recombinant transduced cells. Both the vectors and the recombinant transduced cells can be used therapeutically. The cells can also be used to produce alpha interferon.

### FIELD OF THE INVENTION

This invention relates to gene therapy. More particularly, it relates to retroviral vectors,, such as adenovirus vectors, which express the therapeutically active molecule alpha interferon (“IFN-α”), or therapeutically active portions thereof. Such vectors can be used ex vivo to transfer cells, such as bone marrow or stem cells, which are then reintroduced to a subject suffering from a condition treatable with IFN-α, or can be administered directly, with, e.g., an appropriate carrier, such as a liposome.

### BACKGROUND AND PRIOR ART

Interferon alpha, or “IFN-α”, has been used in a number of ways to treat various conditions that are characterized by cellular abnormalities. It has been found to induce hematological remission in up to 75% of all individuals suffering from chronic myelogenous leukemia, or “CML”. Indeed, it has been shown to induce complete cytogenetic remission in a small number of patients, with gradual selective suppression of malignancies, and restoration of normal hematopoieses. It is to be noted that, when “IFN-α” is used herein, it refers to all of the IFN-60 subtypes, such as IFN-α1, IFN-α5, IFN-α21, and so forth. IFN-α has properties which include anti-viral, antiproliferative, and immunomodulatory activities. In the area of cancer therapy, IFN-α has been used to treat chronic lymphocyte leukemia (Rozman, et al., Blood 71:1295 (1988)); hairy cell leukemia (Quesada, et al., N. Engl. J. Med. 310:15 (1984); Ratain, et al., Blood 65:644 (1985);. Foon, et al., Am. J. Med 80:357 (1986)) as well as metastatic breast cancer, bladder cancer, and malignant melanoma. Additional conditions, outside of the field of cancer, have also been treated with this powerful drug.

Various mechanisms are believed to be involved in the process by which IFN-α functions in these conditions. For example, it is well known that downregulation of adhesion molecule expression plays a critical role in the premature release of malignant, Ph positive blood cells into circulation. It is theorized that IFN-α may reverse this effect, and may also diminish release of immature blood cells. It is also hypothesized that IFN-α's inhibitory effects are mediated, at least in part, through the induction of the so-called “IFN regulatory factor,” and FAS receptor. This latter molecule is known to be expressed on activated cells in hematological malignancies (Hanabuchi, et al., Proc. Natl. Acad. Sci. USA 91:4930 (1994); Owen-Schaub, et al., J. Immunother 14:234 (1993); Robertson, et al., Leukemia Lymphoma 17:51 (1994)). This molecule is also expressed on CD34⁺cells, leading to the assumptions that the antileukemic effect of IFN-α involves enhancing the expression of FAS-R on cells, thereby rendering them more susceptible to cell death.

Nonetheless, it would be incorrect to say that the therapeutic efficacy of IFN-α has been maximized. In large part, this is attributable to the need for long term, continual parenteral administration of the drug in order to maintain therapeutic efficacy.

Gene therapeutic methods appear, in principle, to provide a mechanism by which this necessary, long term continual parental administration could be accomplished. Essentially, gene therapy involves the delivery of a gene which encodes a molecule of interest to a subject, preferably at the site of greatest pertinence. This delivery can be accomplished, for example, by directly administering avector containing the gene via, e.g., injection or aerosol administration, in the form of a composition including a carrier, such as a liposome, or by administering the vector to cells in vitro, followed by reintroduction of the transfected cells to a subject suffering from a disorder amenable to IFN-α treatment.

When considering these forms of therapy, many features and factors have to be considered. Safety is a primary concern. It is important that the vectors being used, while being efficacious, must also not put the patient at risk of other health dangers.

Retroviruses have the ability to introduce and express exogenous DNA in host cell genomes, in a stable way. As a result, retroviruses and retroviruses based vectors have been studied extensively, especially in connection with modifications of mammalian hematopoietic cells. See, e.g., Miller, Blood 76:271-278 (1990); Anderson, Science 256:808-813 (1992), both of which are incorporated by reference. Initial studies in murine models indicated that retroviral vectors based upon murine Maloney leukemia virus can infect long term reconstituting murine stem cells, clinically applicable protocols in both humans, and in large animal models, have been limited, because of very low gene transfer efficiency was achieved. See, e.g., Williams, Hum. Gene Ther 1:229-239 (1990); Abraham, et al., in Hershko, et al., ed, Progress In Iron Research (Plenum Press, 1994, pg. 199-210). Brenner, et al., Lancet 341:85-86 (1993); Brenner, et al., Lancet, 342:1134- 1137 (1993); Krem, et al., Hum. Gene Ther 7:89-96(1996). It is assumed that these low levels may be attributable either to the target cell population, to the retroviral vector construct, or both.

Adenoviral/retroviral mediated gene transfer methods have proven to be successful, both in vitro and in vivo. See, e.g., Chertkov, et al., Stem Cells 11:218 (1993); Abraham, et al., Invest. OphthalmolVis Sci 36:22022 (1995); Cook, et al., ActaHematologica 96:57-63 (1996), all ofwhich are incorporated by reference. Further, co-culture of bone marrow cells with stromal cells has been shown to increase the frequency of gene therapy. See, e.g., Moore, et al., Blood 79:1393-1399 (1992); Nolta, et al., Blood 86:101-110 (1995).

It has recently been shown that fibronectin greatly potentiates retroviral gene transfer into hematopoietic stem cells (Hanenberg, et al., Hum. Gene Ther. 8:2193-2206 (1997), incorporated by reference. This observation is part of the more generalized observation that a critical, first step of gene transfer is the frequency of binding of the retoviral vector on to the target cells. Hence, it is important to have a good technical basis for proceeding with any type of gene therapy.

It has now been shown that it is possible to transduce cells with a retroviral vector, more particularly an adenovirus based vector, that has incorporated therein a nucleotide sequence which encodes an IFN-A, or a portion of an IFN-A molecule that is therapeutically efficacious. These retroviral, IFN-A vectors can be used to transfect or transduce cells, both in vitro and in vivo, leading to production, or enhanced production, of IFN-α. The IFN-α can be used per se; however, a more preferred embodiment of the invention utilizes the retroviral IFN-α vectors in the context of gene therapy, either by the creation of transduced cells ex vivo, which can then be introduced to a subject in need thereof, or by direct introduction of these vectors into the subject.

How these and other features ofthe invention are achieved is set forth in the disclosure which follows.

### DETAILED DESCRIPTION OF PREFERRED EMBODIMENTS

### EXAMPLE 1

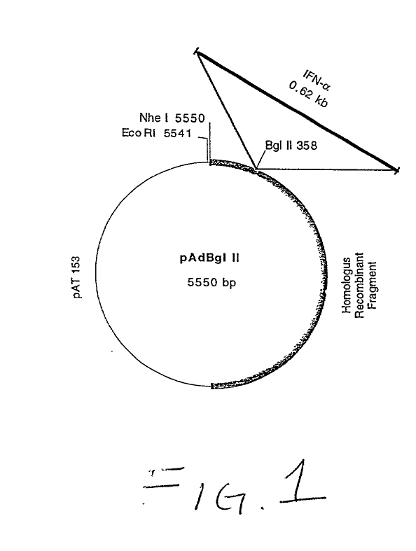

These experiments were designed to examine expression and overexpression of IFN-A in transfected CD34⁺cells. Areplication deficient adenovirus vector which encoded IFN-α (“AdCMV-IFN-α”) was constructed, as was a control vector, i.e., a replication deficient adenovirus vector encoding heme oxygenase -1 (“AdCMV-HO-1”). See Abraham, et al., Inv. Ophthalmol & Vis Sci 36:2202-2210 (1995), incorporated by reference for information on vector preparation, especially for AdCMV-HO-1. In brief, AdCMV-IFN-α was constructed using homologous recombination, where E3 deleted adenovirus dL700 1 and a plasmid encoding human IFN-α (BMG Neo-IFN-α) were used. First, an Xho 1 -Xho 1 fragment of cDNA for human IFN-α was created, and inserted into the Hind III site of commercially available plasmid pRC/CMV, yielding CMV-IFN-α. An NruI-BamHl fragment was generated from this vector, and was inserted into Stul-BamiH1 sites of commercially available vector pBacPac8, to yield pBacPac8-CMV-IFN-α. A BarmHl-Bg1 III fragment containing CMV-IFN-α was excised, and inserted into the Bg1 II site of Padv. Bg1 II, yielding AdCMV-IFN-α. FIG. 1 depicts this construct.

Samples of this construct (10 mg) were digested with EcoRI, and, together with 1 mg of Cla I digested dL7001, referred to supra, were used to cotransfect human embryonic kidney cells (293 cells), which expressed El, using standard methods. Virus was replicated, and then encapsulated into an infectious virus.

Plaque locations were marked on plates after 5 days, and cytopathic effects to the monolayer were observed, microscopically, until they had reached a size appropriate for further processing. Generally, this took about 1 week.

Plaques were then purified via ultracentrifugation using standard methods involving cesium chloride gradients. These were then checked for the presence of cDNA for IFN-α via PCR, and primers specific for the cDNA.

Virus was then released from infected cells 2 days after infection, using five freeze thaw cycles concentrated by centrifugation, and then dialyzed against phosphate buffered saline. Viral titer was determined, using standard methods. In the experiments which follow, the viral titer used was from 1-5×10¹⁰ infectious units/ml.

### EXAMPLE 2

These experiments detail the selection protocols for obtaining pure CD34⁺cells. Heparinized bone marrow cells were aspirated from the posterior iliac crest of either normal volunteers or chronic myelogenous leukemia patients. Low density bone marrow mononuclear cells were then isolated by density centrifugation ofheparinizedmarrow, layered over Ficall-Paque, at 500xg for25 minutes.

These cells were then subjected to sequential counterflow centrifugation elutriation, in accordance with Brandt, et al., J. Clin. Invest 82:1017(1988), followed by sheep erythrocyte rosetting (Verfallie, et al., Blood 77:263(1991)), and immunomagnetic bead depletion (Verfaille, et al., J. Exp. Med 179:509(1990)). In an alternative approach, CD34⁺cells were selected using avidin-biotin immunoadsorption columns, in accordance with Berenson, et al., J. Clin. Invest. 81:951(1988).

The resulting populations were then labelled with anti-CD34 phycoerythrin, and anti-HLA-DR fluorescein isothiocyanate antibodies, and sorted via flow cytometry. The positive cells were used in the experiments which follow.

### EXAMPLE 3

The CD34⁺cells referred to, supra, were resuspended in Iscove's modified Dulbecco's medium, containing 0.5% fetal bovine serum supplementedwithrecombinanthumanIL-6 (50,μml), stem cell factor (100 ng/ml), IL-3 (5ng/ml), G-CSF (50,μ/ml), GM-CSF (50,μ/ml), and M-CSF (10μml)

In a first set of preliminary experiments, varying concentrations of AdCMV-IFN-α (20,40, 80, 120, 160 and 240 pfus/cell) were added directly to the CD34⁺cultures, and coculturing was continued for 4,8, 12 and 24 hour periods. At each time point, samples were removed to determine cell viability, using the propidium iodide method, and for clonal efficiency in methylcellulose culture. The levels of IFN-α MRNA were assessed using RT/PCR.

In parallel, the presence of IFN-α in CD34⁺cell media was determined using a standard, commercially available ELISA. Following 24 hours of incubation, culture medium was removed, and centrifuged at 1000 rpm to remove floating cells. Supernatant was passed through a 0.22μ filter, stored at -70° C., and human IFN-α levels were then determined via the commercial assay.

The results indicated that maximal transfection of the CD34⁺cells was obtained using 120 pfus, with 12 hours of incubation.

Conditioned media from the transfected cells showed elevated levels of IFN-α protein. Specifically, transfected CD34⁺cells produced 4.1±0.6 units of IFN-α per 10⁶ cells over 24 hours, while control cells that had not been transfected produced 0.3±0.1 units under the same conditions, which was identical to the production using AdCMV-HO-1.

With respect to cell viability, the propidium iodide assay indicated that, after 8 hours, the percentage of viable CD34⁺cells was 96.3%, and 94.8% at 12 hours. The percentage of cells which were CD34⁺and maintained membrane integrity was 98.8% at 4 hours, 97.0% at 8 hours, 83.3% at 16 hours, and 73.5% at 24 hours.

### EXAMPLE4

These experiments, as well as the experiments which follow, were designed to determine if long term expression of IFN-α genes in human CD34⁺hematopoietic cells via fibronectin fragment [need word] facilitated retrovirus gene transfer, or modulated its ability on growth and differentiation of colony forming progenitors.

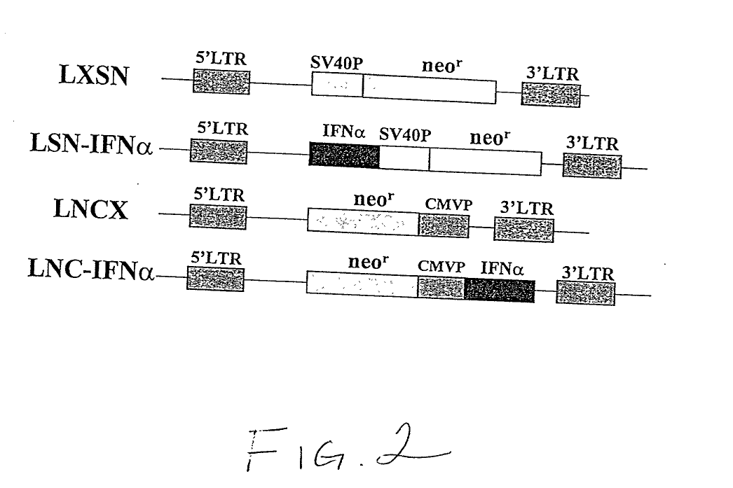

Replication deficient retroviral vectors encoding human IFN-A were constructed. To do so, a 677 base pair XhoI-XhoI human IFNα cDNA fragment was obtained from BMGeoIFN, as taught by Ogura, et al., Cancer Res 50:5702 (1990), incorporated by reference. The fragment was inserted into the XhoI site ofretroviral vector LXSN, to produce LSN-IFN-α. A second retroviral vector was prepared by inserting the IFN cDNA downstream of the CMV promoter of retroviral vector LNCX, to yield LNC-IFNα. FIG. 2 shows the resulting retroviral vectors schematically.

The two retroviral vectors, as well as LXSN and LNCX, were then used to transfect a retroviral packaging cell, PA317, which is commercially available from the American Type Culture Collection. Transfection was carried out using lipofectamine, and standard methods. Transfected cells were cultured, and then viral titer was determined by infection ofNIH 3T3 cells, in accordance with Markowitz, et al., Virology 167:400 (1988), incorporated by reference. A clone using LSN-IFNα (i.e., “PA317/LSN -IFN-α”) produced the highest viral titer, of 1.1×10⁶ colony forming units/ml, and was used in experiments set forth infra.

### EXAMPLE5

Clone PA317/LSN-IFN-α was used to produce virus for transfecting CD34⁺cells. These cells were obtained, as described supra. Aliquots of CD34⁺cells were prestimulated into cell division by incubation for 40 hours with 50 ng/ml of stem cell factor,50 ng/ml of IL-6, and 25 ng/ml of G-CSF, in Iscove's MDM medium supplemented with 20% of fetal bovine serum. The cells were infected for 36 hours, at 37° C., using viral supernatant from the PA317-LSN-IFNα clone, in fibronectin fragment coated 6 well plates. After a 16 hour incubation, the plates were centrifuged for 5 minutes at 1500 rpm, half the media volumes were removed and replaced with fresh medium, followed by an additional 20 hours of incubation.

The CD34⁺cells were then harvested, washed with phosphate buffered saline, and used as indicated in the experiments which follow.

### EXAMPLE 6

A portion of the cells obtained in example 5 were then used in a methylcellulose, semisolid medium for hematopoietic colony assay determinations. In these determinations,3000 CD34⁺cells were plated in Iscove's MDM, supplemented with 0.9% methylcellulose, 30% FBS, 1% bovine serum albumin, 10⁻⁴ M mercaptoethanol, 2mM L-glutamine, 50 ng/ml recombinant stem cell factor, 20 ng/ml of recombinant human GM-CSF, 20 ng/ml of recombinant human IL-3, 20 ng/ml of recombinant human IL-6, 20ng/ml of recombinant G-CSF and 3 units/ml of recombinant human erythropoietin. Cells were incubator at 37° C. in ahumidified atmosphere containing 5% CO₂ in air. Myeloid colonies were defined as comprising at least 50 cells. These were counted as CFU-GM. When more than two clusters of 200 or more hemoglobinized cells formed, these were counted as erythroid BFU-Es.

After 10-15 days, the RNA or DNA of the colonies were removed, and used to measure IFN-α and neo^{r} genes or transcripts, using standard PCR or RT-PCR.

The results indicated that 25-37% of the CFU-GM colonies were positive for neo^{r}.

### EXAMPLE 7

In order to monitor transduction efficiency in the methylcellulose assay, the clonogenic potential of the CD34⁺cells was monitored. In brief, CD34⁺cells were exposed to the viral supernatant in the manner set forth supra. Infected cells were then plated at 3000 cells per dish, which contained 0.9% methylcellulose medium, and multiple growth factors. Individual colonies were picked up after 14 days of plating. Genomic DNA was isolated, and subjected to standard PCR.

In an alternative approach, infected CD34⁺cells wereplated, eitherwith orwithout G418 (0.1−1.2 mg/ml), and colonies were scored after 10-15 days of growth. The percentage of transduced cells was the percentage of G418 resistant cells, and was calculated as the number of G418 resistant colonies, divided by the number of colonies which grew in G418 free medium

The results indicated that the percentage of G418 resistant colonies varied from 31-51%. A total of 41 % of the LSN-IFNA transduced cells acquired substantial resistance to G418 (0.4 mg/ml), as measured by the clonogenic assay. No colony formation was observed when nontransfected cells were treated with G418.

### EXAMPLE 8

In orderto determine the differentiationpotential ofIFNα gene transducedBM CD34⁺cells, and the impact of retroviral infection on the process, clonogenic assays were carried out on control vector (LXSN) transduced, and non-transduced cells. The same assay as described supra was used. The BM CD34⁺cells infected with LSN-IFNa retrovirus (5×10⁵ CFU/ml) generated 246±18 BFU-E, and 203±16 CFU-GM per 3000 plating cells, as compared to 251±24 BFU-E, and 219±20 CFU-GM in control vector transduced CD34⁺cells, as compared to 267±17 BFU-E and 223±20 CFU-GM in non-transduced cells. No significant differences were found in colonogenic formation among the three groups, nor was any difference noted in colony morphology, size, or color.

Further analysis was carried out to assess the integration and transcription of IFNα and the neor genes in cultured MC colonies. PCR and RT-PCR were carried out on DNA and RNA from the colonies. This work demonstrated that IFNα and neo^{r} mRNA were both expressed in LSN-IFN-α transduced colonies, while only neor mRNA was detected in the LXSN colonies. Neither mRNA was found in non-transduced colonies. Similarly, human IFNα and neor gene integration with cellular chromosome DNA were confirmed by PCR from DNA of LSN-IFNα transduced colonies, as compared with LXSN transduced colonies, which were neo^{r} gene positive, and IFNα weakly positive or non-transduced cells, which were only weakly positive for IFNα.

### EXAMPLE 9

In concert with the experiments described supra, the presence of IFNA in various cell media was assayed, using a commercial radioimmunoassay. In brief, following a 24 hour incubation, culture medium was removed, and passed through a 0.45 μM filter. Samples (100 μl) were incubatedwithIFNa specific antibodycoatedbeads for 20hours. Mediumwasremoved, and beads were washed, after which, 200 μml of IFNα antibody I solution was added to each tube, followed by 3 hours of incubation. Reaction mixtures were removed, and samples were washed, three times. The amount of IFNA was determined using a γ counter. Transduced CD34⁺cells with LSN-IFNα secreted IFNα at 72.2±15.4 μml (1 O6 cells per 24 hours), suggesting expression ofthe IFNa gene. CD34⁺cells transected with LXSN did not express significant levels of IFNα (8.3±2.1μml), as compared to non-transduced CD34⁺cells (4.3±1.2 μ/ml).

### EXAMPLE 10

These experiments were designed to assess the effect of transduced cells when used in vivo. Four five-week-old-female, NOD/SCID mice were used. All mice received total body irradiation at 100-150 cGys, after which they were injected, intravenously, with 1×10⁶ human CD34⁺cells that had been transduced with either LSN-IFNα, or, as a control, LXSN. Mice were given multiple injections of human growth hormone intraperitioneally (20 units/mouse/injection), and G-CSF (8μg/mouse/injection), three times a week, as well as GM-CSF and IL-3 (6μg/mouse/injection), twice a week.

Mice were sacrificed 15, 60, 90 and 180 days after transplantation. Cellular contents of both femurs and both libias were flushed, and RNA and genomic DNA were extracted, using standard methods. RC-PCR was carried out, as described supra, to determine IFNα and neortranscript. The results are presented in the table which follows. Note that, over a period from 2 weeks through 60 days after transplantation, 100% of the mice which had been engrafted with LSN-IFNa expressed neo^{r} mRNA in bone marrow. Genetically modified CD34⁺cells which had been transduced with LXSN showed similar levels of neor mRNA, as compared to those engrafted with LSN-IFNα transduced cells.

At 90 and 180 days, 66.7% of the mice transduced with the LSN-IFN-α were positive for IFN-α and neor transcripts. The drop from 100% can be attributed to a need to better defme the cytokine requirements of the engrafted cells.

TABLE 1
Detection of IFN-α and Neo^{r} transcripts in BM cells of NOD/SCID mice
transplanted with retro virus-transduced human CD34⁺ cells

| No. of | Time | Retroviral | IFNα (⁺) | Neo^{r} (⁺) |
|---|---|---|---|---|
| mice | (month) | vectors | Number (%) | Number (%) |
| 6 | 0.5 | LSN-IFNα | 6 (100) | 6 (100) |
| 2 | 0.5 | LXSN | 0 (0) | 2 (100) |
| 8 | 2 | LSN-IFNα | 6 (75.0) | 8 (100) |
| 1 | 2 | LXSN | 0 (0) | 1 (100) |
| 9 | 3 | LSN-IFNα | 6 (66.7) | 7 (77.8) |
| 2 | 3 | LXSN | 0 (0) | 2 (100) |
| 6 | 6 | LSN-IFNα | 4 (66.7) | 4 (66.7) |
| 3 | 6 | LXSN | 0 (0) | 2 (66.7) |

### EXAMPLE 11

In these experiments, the studies on transfection of cells were extended to N1H 3T3 and K562 cells. Both are commercially available lines.

The retroviral vectors described supra, i.e., LSN-IFNA, LNC-IFNα, LXSN and LNCX, were used, as were the packaging lines PA 317 and PG 13. The packaging cells were seeded in 60mm culture wells, at 2×10⁵ cells, and incubated for 24 hours, after which attached cells were washed, twice with serum reduced minimal essential medium. Cells were then combined with 5 μg of retroviral vector, incubated in 20 μl of lipofectamine, for 30 minutes, at ambient temperature. The mixture was incubated at 37° C. for 5 hours, after which 3 ml of DMEM containing 20% FBS was added. This was incubated for 18 hours, after which the medium was replaced, and incubated for 24 hours. Cells were passaged, 1:10, into medium containing G418 (600,μg/ml), following 14 days of culture (37° C., 5% CO₂), cells were subjected to single cell cloning. The G418 resistant clones were isolated, and tested for potential to transcribe IFN-α, and neo^{r} mRNA using standard Northern blotting or RT-PCR, as described supra. Further, the cells were tested for their ability to secrete IFN-α into culture medium via a standard radioimmunoassay, or by determining the inhibitory effect of the medium via a cytopathic effect assay using HeLa cells and vesicular stomatis virus, as described by Zhang, et al., Proc. Natl Acad. Sci USA 93:4513 (1996), Geng, et al., Cytokines Mol. Ther 1:289 (1995), and Rubenstein, et al., J. Virol 37:755 (1981), all of which are incorporated by reference.

All LSN-IFN-α transduced PA317 clones, LNC-IFN-α transduced PG 13 clones, and LNC-IFN-α transduced PA 317 clones produced detectable amounts of IFN-α (LSN-IFNα PA 317: 232.9±199.6; LNC-IFN-α PA 13: 95.8±14.2; LNC-IFN-α PA 317: 101 ±11.7 ,μ/ml/10⁶ cells/24 hours). One clone, i.e., PA 317/LSN-IFNα-C3, was an especially good producer (475±50.2 μ/ml/10⁶ cells/24 hours), and was used in the experiments which follow. This line also had a viral titer of 1.1×10⁶ CFU/ml.

### EXAMPLE 12

NIH 3T3 cells were transfected with supernatant obtained from PA 317/LSN-IFN-α -C3, in accordance with Markowitz, et al., Virology 167:400 (1988). This is how CFUs, reported supra, were determined.

Transduced K562 cells (K562LSN-IFN-α and K562/LXSN) were obtained by infecting K562 cells in the same way, following two weeks of culture in G418 containing media.

The levels oftranscription of IFNA and neor genes from each group of transduced cells were determined, using standard RT-PCR. All cell types were able to transcribe IFN-α genes.

### EXAMPLE 13

Further experiments were carried out to determine if secreted IFN-α was biologically active and could inhibit K562 cell growth. To do this, K562 cells were seeded into 12 well culture plates (1×10⁵ cells/well), and grown for 1, 2, 3 or 4 days in a standard culture medium. Cell suspensions (50μl) were mixed with an equal volume of 0.4% trypan blue, and allowed to stand for 2-3 minutes at ambient temperature. Cell viability was then calculated as the percentage of viable, or unstained cells, as compared to total number of cells.

In additional experiments, supernatants from PA 317, PA 317/LXSN and PA 317/LSN-IFN-α were added to 12 well plates, each of which contained 2’10⁵ K562 cells. These were incubated for 1 hour, or 1, 2, 3 or 4 days. Cell viability was determined, as described supra, and cell proliferation determined using standard methods.

It was found that K562 cell proliferation was effectively suppressed when PA 317/LSN-IFN-α supernatant was added, with a 42.6% decrease in absorption value after 4 days. There were no significant differences between noninfected K562 cells, and cells infected with PA 317/LXSN supernatant.

Partial inhibition of K562 was followed by infection with the PA 317/LSN-IFN-α supernatant. Further, cell/cell aggregation was observed in IFN-α transduced cells.

In experiments not reported here, constructs were prepared using murine IFN-α. Murine bone marrow cells transfected with this construct behaved in a similar fashion in that they generated lineages, i.e., erythroid and myeloid as above. Genetically modified bone marrow cells support transplantation, survival and entraftment in irradiated mice.

### EXAMPLE 14

The ability of supernatant of PA 317/LSN-IFN-α to modulate apoptosis in transduced K562 cells was studied, following Nicoletti, et al., Immunol. Meth. 139:271 (1991). To elaborate, cultured cells were harvested fixed in 2 ml cold 70% ethanol at 4° C. for 1 hour, washed in 5 ml PBS, and resuspended in 0.5 ml PBS. Samples (0.5 ml) were mixed with 0.5 ml RNA se (1 mg/ml in PBS), followed by mixing with 0.1 ml propidium iodide (100 mg/ml in PBS). Mixed cells were incubated in the dark for 15 minutes, and then analyzed for cell cycle chromosome DNA distribution (percentages of cells in GO+GI, 5, and G2 +M phases of the cell cycle, and apoptolic cell death was observed using a flow cytometer. Further, morphologic changes in cellular nuclei were observed via fluorescence microscopy following fixing of stained cells. The cells were exposed to the supernatants for 12, 34 and 58 hours.

The result indicated that there was no apparent apoptosis, although there was marked S phase elevation, accompanied by reduction of G2 and M phase after 58 hours of exposure to the retroviral supernatant. Morphological changes typical of apoptosis were not seen.

### EXAMPLE 15

These experiments were designed to determine if IFN-α gene transfer could affect adhesion molecule expression. To do this, K562 cells transduced with LSN-IFN-α, K562 cells transduced with K562, and wild type K562 were tested. The cells were harvested, washed with PBS containing 0.05% NaN₃, 0.1% BSA, and 0.1 g1L CaCl₂/MgCl₂. Cells were then incubated with 50μl of 1:1000 dilutions of murine MAb to VLA/Mac-1, for 1 hour, at 4° C. Cells were then washed with the solution referred to supra, and incubated for 30 minutes with goat, anti-mouse FITC conjugated antibody at 4° C., in the dark. Cells were washed once more with the above solution, and then with a solution of PBS containing, 0.05% NaN₃, and 0.1g/1 of Ca Cl₂/Mg C1₂. Cells were fixed in 4% paraformaldehyde in PBS for 10 minutes, and then analyzed using a flow cytometer.

Similarly, cells were tested in L-selection using the above protocol, but a 1:400 dilution (50μml) of FITC—conjugated L-selection MAB.

Results indicated that VLA-4Mac-1 were upregulated in the cells transduced with the IFN-α cDNA, but not the others. No increase in L selection expression was observed.

### EXAMPLE 16

The differentiation capability of CD34⁺cells transduced with retroviral vectors was determined. Aliquots of CD34+cells were prestimulated into cell division via incubation, for 40 hours, with stem cell factor (50 ng/ml), IL-6 (50 ng/ml), and G-CSF (25 ng/ml), in IMDM medium supplemented with 20% FBS. Dividing cells were infected for 36 hours, at 37° C., with viral supernatants, in fibronectin plates as described supra. Cells were harvested following infection, washed twice with PBS, and then were either plated in methylcellulose, as described supra, or allowed to grow in suspension culture.

Those cells plated in methylcellulose were plated in IMDM supplemented with 0.9% methylcellulose, 30% FBS, 1% BSA, 10⁻⁴ M 2-mercaptoethanol, 2mM L-glutamine, 50 ng/ml recombinant human stem cell factor, 20 ng/ml of recombinant human GM-CSF, 20 ng/ml of recombinant human IL-3, 20 ng/ml of recombinant human IL-6 20 ng/ml of G-CSF and 3μ/ml of recombinant human erythropoietin. The CD34⁺were incubated at 37° C. in a humidified atmosphere containing 5% CO₂ in air. CFU-GMs and BFU-Es were counted, as described supra. After 10-15 days, RNA and genomic DNA were extracted from colonies, and RT-PCR was carried out (to measure IFN-α and neor transcript), or PCRwas carried out (to analyze integration of IFN-α genes).

The expression of IFN-α genes, and integration into colonies were confirmed using the RT-PCR and PCR, but no IFN-a expression or integration was found in either LXSN transduced cells, or controls. The LSN-IFN-α transduced cells generated 246±18 BFU-E per 3000 plating cells, as compared to 251±24 in LXSN transduced cells, and 267±17 BFU-E in controls.

The foregoing examples elaborate upon the invention which involves, inter alia, recombinant retroviral vectors which comprise, in operable linkage with a promoter, a nucleic acid molecule that encodes an IFN-α molecule or a therapeutically active portion thereof, as it is well known that in many cases, proteins of a molecule will be sufficient, or even more active than the whole molecule.

Preferably, the retroviral vector is one which includes a sequence encoding for a selection marker, such as neomycin resistance, and a promoter sequence, such as an SV40 or CMV promoter. Other promoters, be they constitutive or inducible, as well as other marker sequences, may be used. These can include, e.g., other survival markers, such as sequences conferring resistance to methotrexate, or signalling markers, such as a sequence encoding green fluorescence protein, or some other indicia that transfection has occurred.

These vectors are used to create transduced cells, which are another feature ofthe invention. These transduced cells are recombinant cells, preferably eukaryotic, which have been treated so that the retroviral vectors are integrated into their genome. Exemplary, but by no means the only type of cell which can be so used, are bone marrow cells, stem cells, natural killer cells, dendritic cells, stromal cell layers, and so forth. Bone marrow and stem cells are especially preferred.

Both the retroviral vectors and the recombinant cells described herein can be used in a further facet of the invention, which is the treatment of a subject suffering from a disorder responsive to IFN-α, by administration to said subject of a retroviral, IFN-α vector in an amount sufficient to produce IFN-α in said subject. In one embodiment ofthe invention cells are transduced ex vivo, and then introduced to the subject. Preferably, these are autologous cells of a type related to malfunctioning or dysfunctional cells of the subject. In the case of leukemias, such as those described supra, bone marrow cells are preferred, but other cell types can be used. Further, the retroviral vectors can be administered, “as is,” or in the form of a composition which increases the ability of cells to take up and to integrate the vectors. Liposomes are one example of the components which can be combined with the vectors to facilitate their uptake.

Preferably, the cells and/or retroviral vectors are administered in a way which delivers them directly to a site where therapy is most advantageous. For example, bone marrow transplantation is an effective way to administer materials when the condition being treated is leukemia, and other modes of administration, including intravenous or intramuscular inj ection, intraperitoneal or topical administration, aerosol or topical application, and so forth, are all approaches that can be taken.

While leukemia is one condition which can be treated in this way, any condition for which IFN-α is an effective agent can be treated in this manner. Exemplary of other such conditions are other blood related neoplasias, such as non-Hodgkin's lymphoma, neoplasias, such as multiple myeloma, infections, such as viral infections including hepatitis B and C infections, and autoimmune disorders such as multiple sclerosis. The artisan of ordinary skill will recognize that these are exemplary ofdisorders that can be treated with the retroviral vectors, but it is not an inclusive listing.

Also a part ofthe invention is the use ofthe recombinant, transduced cells to produce IFN-α. As was shown, supra, the recombinant cells of the invention produce large amounts of IFN-α. This can be purified from culture medium, and used in any of the ways in which IFN-α is used in the art

Yet a further feature of the invention is the use of so-called “naked DNA,” i.e., non-vector incorporated DNA that encodes interferon alpha, for the treatment of conditions such as leukemia, and the other conditions elaborated upon herein which are treated using the retroviral constructs of the invention.

Other features of the invention will be recognized by the skilled artisan, and need not be set forth here.

The terms and expressions which have been employed herein are used as terms of description and not of limitation, and there is no intention in the use of such terms and expressions of excluding any equivalents of the features shown and described or portions thereof, it being recognized that various modifications are possible within the scope of the invention.

We claim:

1. An isolated, recombinant retroviral vector comprising a nucleic acid molecule that encodes interferon alpha, in operable linkage with a promoter.

2. The isolated, recombinant retroviral vector of claim 1, wherein said promoter is an SV 40 promoter.

3. The isolated, recombinant retroviral vector of claim 1, wherein, said promoter is a cytomegalovirus promoter.

4. Recombinant cell comprising the isolated, recombinant retroviral vector ofclaim 1 integrated into the genome of said cell.

5. The recombinant cell of claim 4, wherein said cell is a bone marrow cell, a stem cell, a dendritic cell, a cell from a stromal cell layer,or a natural killer cell.

6. A method for treating a subject suffering from a condition against which interferon alpha is effective, comprising administering to said subject an amount of the isolated, recombinant retroviral vector of claim 1 sufficient to integrate into cells of said subject and to produce interferon alpha in an amount sufficient to alleviate said condition.

7. The method of claim 6, wherein said condition is a neoplasia.

8. The method of claim 7, wherein said neoplasia is a leukemia.

9. The method of claim 8, wherein said leukemia is CML.

10. The method of claim 9, wherein said leukemia is non-Hodgkin's lymphoma.

11. The method of claim 6, wherein said condition is a hepatitis B infection, a hepatitis C infection, or multiple sclerosis.

12. A method for treating a subject suffering from a condition against which interferon alpha is effective, comprising administering an amount of the recombinant cell of claim 4sufficient to alleviate said condition.

13. The method of claim 12, wherein said condition is a neoplasia.

14. The method of claim 13, wherein said neoplasia is a leukemia.

15. The method of claim 14, wherein said leukemia is CML.

16. The method of claim 12, wherein said condition is a hepatitis B infection, a hepatitis C infection, or multiple sclerosis.

17. A method for producing an IFN-α comprising culturing the recombinant cell of claim 4 for a time sufficient to produce IFN-α, and purifying said IFN-α.

18. A method for treating a subject suffering from leukemia, comprising administering to said subject an amount of a nucleic acid molecule consisting of a nucleotide sequence which encodes interferon alpha sufficient to integrate into the genome of leukemia cells and to produce a therapeutically effective amount of interferon alpha.

19. The method of claim 18, wherein said leukemia is CML.

20. The method of claim 18, wherein said leukemia is non-Hodgkin's lymphoma.
